(19) United States
(12) Patent Application Publication
(10) Pub. No.: US 2014/0090651 A1
(43) Pub. Date: Apr. 3, 2014

(54) ABDOMINAL WRAP WITH WINDOW

(71) Applicant: Tunisia Melendez, Colchester, CT (US)

(72) Inventor: Tunisia Melendez, Colchester, CT (US)

(21) Appl. No.: 14/032,545

(22) Filed: Sep. 20, 2013

Related U.S. Application Data
(60) Provisional application No. 61/703,501, filed on Sep. 20, 2012.

(51) Int. Cl. A61B 19/02

(52) U.S. Cl. CPC A61B 19/026
USPC 128/846

(57) ABSTRACT

A medical wrap configured to be secured about a body or torso of a patient, or around a limb or other portion of the patient. The wrap comprises a main body of stretchable fabric. The wrap has a first fastener. The wrap has an opening formed in the main body. The opening has a flap and a second fastener selectively securing the flap to the main body.

The present application claims priority to Provisional Patent Application No. 61/703,501 filed by applicant on Sep. 20, 2012.

### FIELD OF THE INVENTION

The present invention relates generally to medical binders or wraps. More specifically, the present invention is a wrap that includes one or more of a window, a flap, or a pocket designed to shield, retain, and provide a caregiver access to medical paraphernalia such as IV tubing, drainage bulbs, valves and the like in place and away from a patient's reach. The present invention is further designed to receive medical paraphernalia in a pocket which protects the patient's skin from that paraphernalia.

### BACKGROUND OF THE INVENTION

Hospital patients often have intravenous tubing, feeding tubes, or drainage devices inserted and left in place for a long period of time while they convalesce. Patients with a traumatic brain injury (“TBI”) may move involuntarily and catch hold of such medical paraphernalia. Such medical paraphernalia may thereby be damaged, removed, or contaminated, increasing the risk of infection and possibly requiring further surgery or re-insertion. An abdominal binder is an elastic wrap placed snugly around a patient to prevent movement of such medical paraphernalia. One example is sold under model no. TN 30-45 by DeRoyal Industries (Powell, Tenn.).

A perceived problem with the current abdominal binder is that it is a single wrap with a single point of attachment. In order to access the medical paraphernalia beneath, a caregiver must open the entire binder. This may disturb a resting patient and may result in disruption of the paraphernalia. Further, the abdominal binder must be snug in order to secure the medical paraphernalia; however this presses the medical paraphernalia into the skin and can cause skin breakdown, irritation, and infection.

Accordingly, a need exists for an improved medical wrap that addresses the above-described disadvantages of conventional binders.

### SUMMARY OF THE INVENTION

It is an object of the present invention to provide a wrap. The wrap is securable about a body or torso of a patient, or around a limb or other portion of the patient. The wrap comprises a main body of stretchable fabric. The wrap has a first fastener. The wrap has an opening formed in the main body. The opening has a flap and a second fastener selectively securing the flap to the main body.

The present invention is a wrap with a flap or window that can be opened without disturbing placement of the entire wrap about a portion of a patient's body. The window further comprises a liquid-resistant pouch sized to accept one or more of a plurality of medical paraphernalia and into which the medical paraphernalia may be placed in order to keep them organized and keep them shielded from the skin. The invention offers a dual benefit of easier access to the medical paraphernalia by caregivers and also keeping the skin protected.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Description of the Embodiments, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1 illustrates a side view of a wrap fastened around a patient's torso in accordance with an embodiment of the present invention;

FIG. 2 illustrates a is a side view of the wrap disposed behind the patient's torso showing a pouch in accordance with an embodiment of the present invention;

FIG. 3 illustrates a side view of the wrap fastened around the patient's torso with a flap open of the present invention;

FIG. 4 illustrates a pouch of the flap in accordance with an embodiment of the present invention;

FIG. 5 illustrates a schematic side view of the wrap showing a plurality of ventilation elements of the present invention;

FIG. 6 illustrates a perspective view of the flap showing a second flap of the present invention;

FIG. 7 illustrates a perspective view of the flap around a patient's torso with the flap in the open or detached position;

FIG. 8 illustrates a perspective view of the flap around a patient's torso with the flap in the closed, fixed or attached position;

FIG. 9 illustrates a perspective view of the flap around a patient's torso with the flap in the open or detached position;

FIG. 10 illustrates a perspective view of the flap around a patient's torso with the flap in the open position;

FIG. 11 illustrates a perspective view of the flap around a patient's torso with the flap in the closed, fixed or attached position;

FIG. 12 illustrates a perspective view of the flap around a patient's torso with the flap in the open or detached position;

FIG. 13 illustrates a perspective view of the flap around a patient's torso with the flap in the closed, fixed or attached position; and

FIG. 14 illustrates a perspective view of the swooped neck feature of a flap having the wrap around a patient's torso in the open or detached position.

### DESCRIPTION OF THE EMBODIMENTS

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

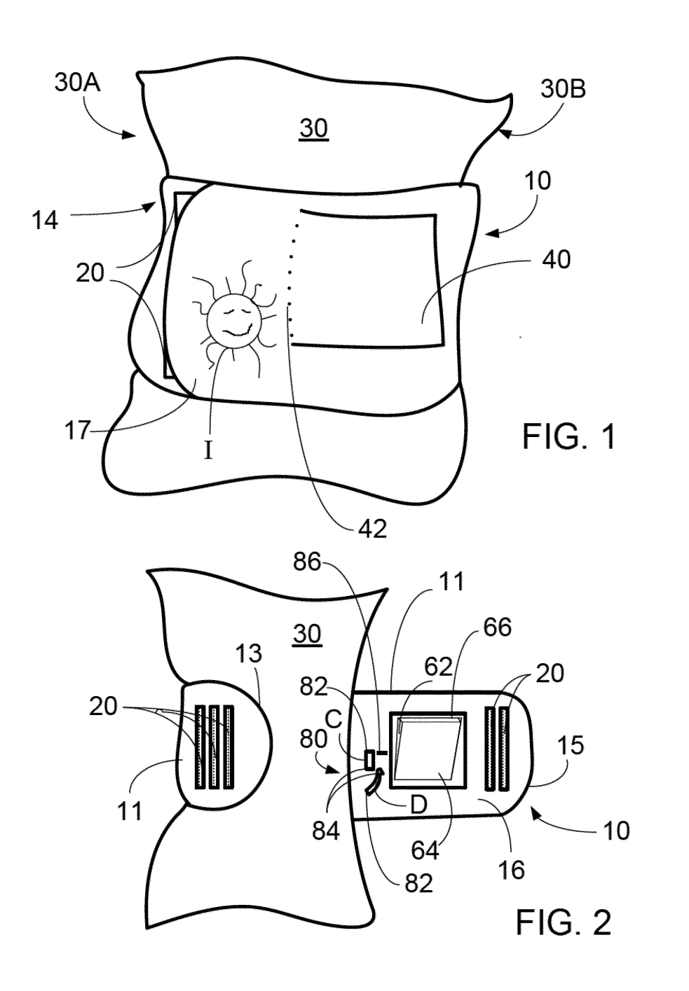

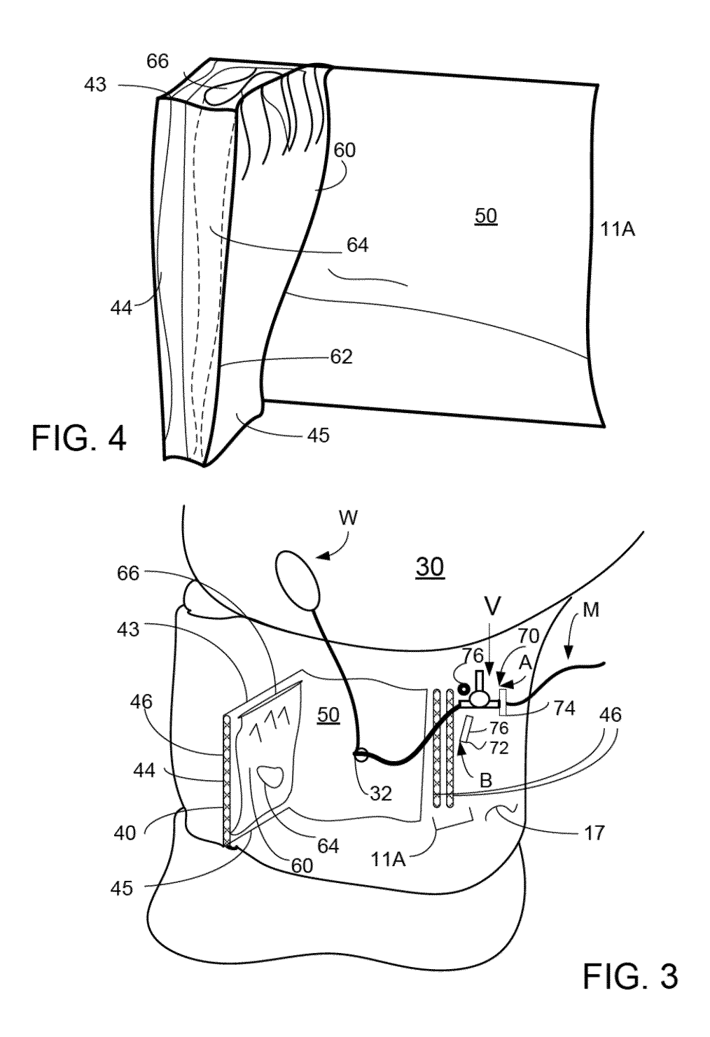

The drawings featured in FIGS. 1 through 14 are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

Referring to FIG. 1, a wrap 10 is disposed around a portion of a patient's body, e.g. a torso 14 of the patient, generally indicated by the numeral 30. For the purposes of the figures herein, the numeral 30A is indicating the ventral side of the patient 30 and the numeral 30B is directed at the dorsal side of the patient 30. A first fastener 20 of the wrap 10 selectively secures the wrap 10 in place about the patient 30. In one embodiment, the wrap 10 is oriented so that, when secured about patient 30, a flap 40 is located at or near the left iliac region of patient 30. In another embodiment, it is understood that the flap 40 is oriented to cover a relevant portion of the patient 30 (e.g. an area of treatment as described below). It should be appreciated that in various embodiments the wrap 10 may be of various lengths or adjustable in length to surround patients having torsos of varying sizes.

The figures herein demonstrate the wrap 10 oriented to the left side of the patient 30 and it is understood that this orientation could be changed for different embodiments and different applications. The present invention may be different sizes to accommodate other parts of the body, for example a head, thigh, or arm, or a pediatric size. Additionally, it is within the scope of the present invention to use the wrap 10 to surround other regions of a patient including the head, torso, limbs, or other portions thereof. The present invention is not limited to human medicine and it is anticipated that the benefits described herein are applicable in veterinary medicine, including equine, canine, feline, and other companion animal applications. Similarly, the term “patient” should be construed broadly, including for example, the patient may be a human or animal.

The wrap 10 is comprised of a main body portion 11 that is stretchable. Although other alternatives are understood to be commonly available, in one embodiment the main body portion 11 is a cotton-polyester-spandex blend fabric. In another embodiment, the main body portion 11 is needle-punched cotton felt to permit selective attachment and detachment of the wrap 10 about the patient 30. In one embodiment, the material is washable and can be sanitized for repeated uses in medical treatment. In another embodiment, the wrap is intended to be used once and discarded once soiled.

As shown in FIG. 2, in one embodiment, the main body portion 11 is generally rectangular. The main body portion 11 includes a first end 13, and a second end 15 disposed opposite the first end 13. The main body portion 11 includes an inner surface 16 that contacts the patient 30 opposite an outer surface 17. At least the main body portion 11 is comprised of stretchable fabric. The main body portion 11 is of sufficient length to wrap around a desired portion of the patient 30 and secure to itself by means of the first fastener 20. In one embodiment, the main body portion is shaped or adapted such that it may wrap around, for example, a head, neck, joint, or appendage of the patient 30.

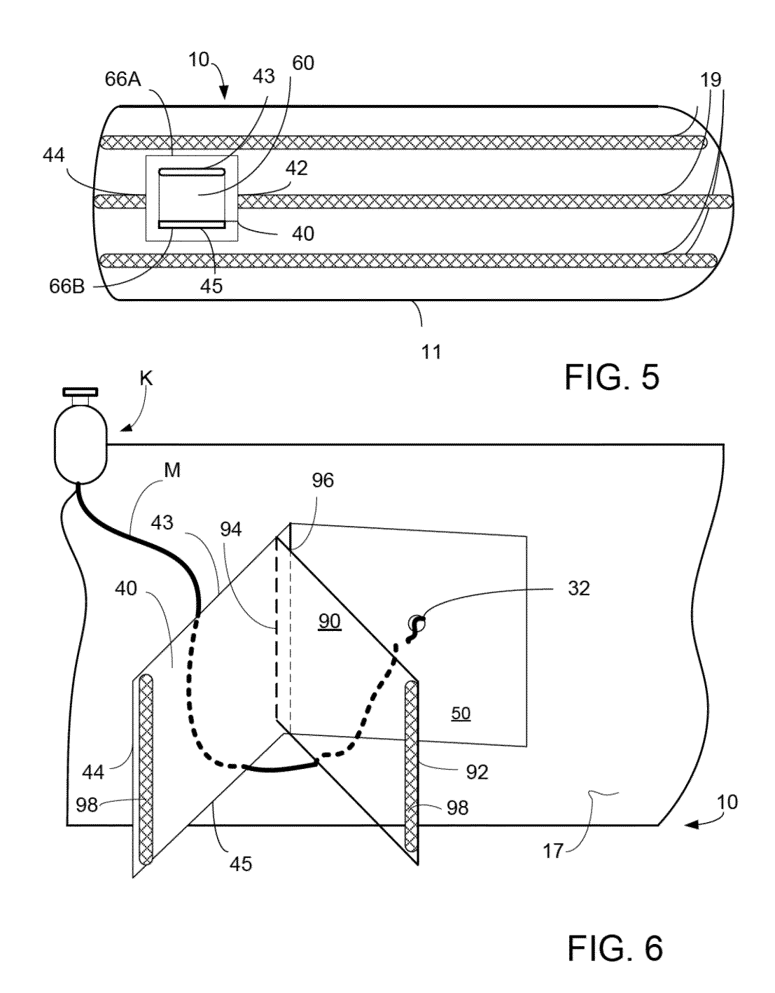

As shown in FIG. 1, in one embodiment there is at least one of a plurality of indicia I on the outer surface 17. The indicia I may include text or graphic elements designed to lift the spirits of the convalescing patient, a brand name or promotional message, or the like. In one embodiment, the main body portion 11 is a contiguous piece of stretchable fabric. In another embodiment shown in FIG. 5, the wrap 10 is assembled with a plurality of ventilating elements 19. For example, in one embodiment, the ventilating elements 19 are interlaced latex-free knit elastic. In another embodiment, the ventilating elements 19 are a plurality of panels of moisture wicking polyester, such as, for example COOLMAX® fabric (a trademark of Invista North America of Wichita, Kans.). The ventilating elements are shown in FIG. 5 disposed lengthwise along the main body portion 11 of the wrap 10. However, it is understood the ventilating elements 19 may be oriented perpendicular or orthogonal relative to the length of the wrap 10 at periodic locations along the length of the main body portion 11, or along substantially the entire length of the main body portion 11.

As shown in FIG. 2, the first end 13 and the second end 15 of the wrap 10 include mating components of the first fastener 20. In one embodiment, the first fastener 20 is a hook and loop closure (e.g., Velcro™, a trademark of Velcro Industries B.V. LLC, Amsterdam, Netherlands) where a first component of the closure, e.g. the loop, is disposed at the first end 13 and a second component of the closure, e.g. the hook, is disposed at the second end 15. In another embodiment, the first fastener 20 is attached to the second end 15 and attaches directly to the outer surface of the main body of the wrap 10. In another embodiment, the first component of the first fastener 20 is a male end of one or more snaps and the second component of the closure is a female end of a corresponding one or more snaps. In another embodiment, the first component of the first fastener 20 is one or more bars closures to engage with one or more hooks of the second component of the closure. It is understood that a variety of sizes of main body portion 11 may be required for patients of different sizes and that having a first fastener 20 that may accommodate different fits is desirable, such as, for example, having multiple fastening points for the first component of first fastener 20.

Referring to FIG. 3, in one embodiment, the outer surface 17 has at least one of a plurality of first tabs 70 that may selectively receive a tube or other medical paraphernalia, generally indicated by the letter M. Each of the first tabs 70 has a first end 72 opposite to a second end 74. In one embodiment, the second end 74 is fixedly attached to the outer surface 17. In another embodiment, the second end 74 is selectively attached to the outer surface 17, for example, by hook and look closure (e.g., Velcro™, a trademark of Velcro Industries B.V. LLC, Amsterdam, Netherlands). The first end 72 has a third fastener 76 which allows the first end 72 to selectively attach and detach from the outer surface 17. As shown in FIG. 3, a pair of first tabs 70 are arranged on either side of a Lopez valve, indicated generally by the letter V. The first tab indicated by the letter A is in the closed, fixed or attached position; the first tab indicated by the letter B is in the open or detached position.

Referring to FIGS. 1 and 3, a flap 40 is formed in the main body portion 11 of the wrap 10 and completely covers an opening 50. As should be appreciated, the opening 50 may reveal and provide access to an area of treatment, e.g. the insertion point 32 of a tube or medical paraphernalia, on the body of the patient 30 and/or medical paraphernalia used to treat that patient. In one embodiment, the flap 40 is comprised of the same stretchable fabric as the main body portion 11 of the wrap 10. In another embodiment, the flap 40 is contiguous with the main body portion 11 of the wrap at a hinge or seam 42. In another embodiment, the flap 40 is an inelastic material. In another embodiment, the flap 40 is a highly elastic material that may expand and/or contract readily and independently of the expansion and contraction of the main body portion 11.

Referring to FIG. 1, the flap 40 is attached by the seam 42 to the main body portion 11 of the wrap 10. In one embodiment, the seam 42 is a fold in the material of the main body of the wrap 10. In another embodiment, the seam 42 is sewn attachment between the material of the flap 40 and the main body portion 11 of the wrap 10. In another embodiment, the seam 42 is an adhesive or bonded attachment between the material of the flap 40 and the main body portion 11 of the wrap 10.

Referring to FIG. 3, in one embodiment the flap 40 is a rectangular shape. The flap 40 has an end 44 opposite to the seam 42. The flap 40 is further defined by a top edge 43 and a bottom edge 45 which are substantially parallel to each other and substantially perpendicular to the seam 42

Referring to FIG. 3, the flap 40 has a second fastener 46, for example, at the end 44 which allows the flap 40 to selectively attach and detach from the main body portion 11 of the wrap 10. In one embodiment, shown in FIG. 3, the second fastener 46 is a hook and loop closure. As shown in FIG. 3, the second fastener 46 is at the end 44 and an area 11A proximate to the opening 50. In another embodiment, the second fastener 46 extends along at least a portion of the top edge 43. In another embodiment, the second fastener 46 extends along at least a portion of the bottom edge 45. As shown in FIG. 3, the second fastener 46 attaches directly to the main body portion 11 of the wrap 10. In another embodiment, the second fastener 46 comprises a hook and loop closure affixed to the main body of the wrap 10. In another embodiment, the first component of the second fastener 46 is a male end of one or more snaps and the second component of the closure is a female end of a corresponding one or more snaps. In another embodiment, the first component of the second fastener 46 is one or more bars closures to engage with one or more hooks of the second component of the closure.

In another embodiment illustrated in FIG. 2, the inner surface 16 has one or more of a plurality of second tabs 80 that may selectively receive a tube or other medical paraphernalia. Each of the second tabs 80 has a first end 82 opposite to a second end 84. In one embodiment, the second end 84 is fixedly attached to the inner surface 16. In another embodiment, the second end 84 is selectively attached to the flap 40, for example, by hook and look closure (e.g., Velcro™, a trademark of Velcro Industries B.V. LLC, Amsterdam, Netherlands). The first end 82 has a fourth fastener 86 which allows the first end 82 to selectively attach and detach from the inner surface 16. The second tab indicated by the letter C is in the closed, fixed or attached position; the second tab indicated by the letter D is in the open or detached position. In another embodiment, there are one or more of the second tabs 80 selectively attached to the flap 40.

As shown in FIGS. 2, 3, and 4, a pouch 60 is disposed on or formed within the flap 40. The pouch 60 includes an expandable wall 62 defining an interior cavity 64 therein. The interior cavity 64 is accessible by one or more openings 66 in pouch 60. As shown in FIG. 3, a bulb drain, generally indicated by the letter W, may be inserted into the opening 66 and rest in the interior cavity 64. As shown in FIGS. 2 and 3, the opening 66 is at the top edge 43 of flap 40, however it is understood that the opening 66 could be at or near the seam 42, the end 44, or the bottom edge 45 of the flap 40. Further, there could be one or more openings 66 at or near one or more of the seam 42, the top edge 43, the end 44, and the bottom edge 45. In one embodiment, as shown in FIG. 5, the first opening 66A is at the top edge 43 and the second opening 66B is at the bottom edge 45, permitting the medical paraphernalia to rest in the interior cavity 64.

Referring FIG. 6, in another embodiment, the interior cavity 64 is selectively formed by a second flap 90 has a first end 92 and a second end 94. As shown in FIG. 6, the second end 94 is fixedly attached to the flap 40 by a seam 96. In one embodiment, the seam 96 is a sewn attachment between the material of the flap 40 and the second flap 90. In another embodiment, the seam 96 is an adhesive or bonded attachment between the material of the flap 40 and the second flap 90. The first end 92 has a fastener 98. In one embodiment as shown in FIG. 6, the fastener 98 is a hook and loop closure (e.g., Velcro™, a trademark of Velcro Industries B.V. LLC, Amsterdam, Netherlands) where a first component of the closure, e.g. the loop, is disposed at the first end 92 and a second component of the closure, e.g. the hook, is disposed at the flap 40. The medical paraphernalia inside the interior cavity 64 may be accessed by selectively detaching the fastener 98 from the flap 40. In one embodiment, as shown in FIG. 6, the medical paraphernalia is connected to, for example, a kangaroo pump and bag, generally indicated by the letter K. It should be appreciated that while the second flap 90 is shown as opening at the first end 92 about the seam 96 at the second end 94; in another embodiment the second flap 90 may open about a seam proximate to the bottom edge 45 of the flap 40. In another embodiment, the second flap 90 may open about a seam proximate to the end 44 of the flap 40. In another embodiment, the second flap 90 may open about a seam proximate to the top edge 43 of the flap 40.

Referring again to FIGS. 2, 3, and 4, in one embodiment, the expandable wall 62 is comprised of stretchable fabric such that the interior cavity 64 may be selectively enlarged and reduced to accommodate medical paraphernalia disposed therein. In another embodiment, the pouch 60 is made of an inelastic liquid-resistant material, for example a denier nylon taffeta that is coated with polyurethane. In another embodiment, the pouch 60 is an inelastic liquid-resistant material and the flap 40 is elastic such that medical paraphernalia, e.g. a drainage device (not shown) which expands over time, may expand away from the patient. The benefit of the inelastic pouch 60 in conjunction with the elastic flap 40 is that a medical device does not become pressed into the skin of patient 30. It is widely understood that pressure on the skin over time can lead to skin irritation, skin breakdown, increase the risk of infection, and provide discomfort to the patient.

One benefit of a liquid-resistant material for pouch 60 is keeping moisture from leaking or spilling medical paraphernalia away from the skin of patient 30. It is widely understood that moisture on the skin of a patient can lead to skin irritation, skin breakdown, increase the risk of infection, and provide discomfort for the patient. Further, in one embodiment, the main body portion 11 of wrap 10 is a breathable fabric which provides a moisture wicking effect.

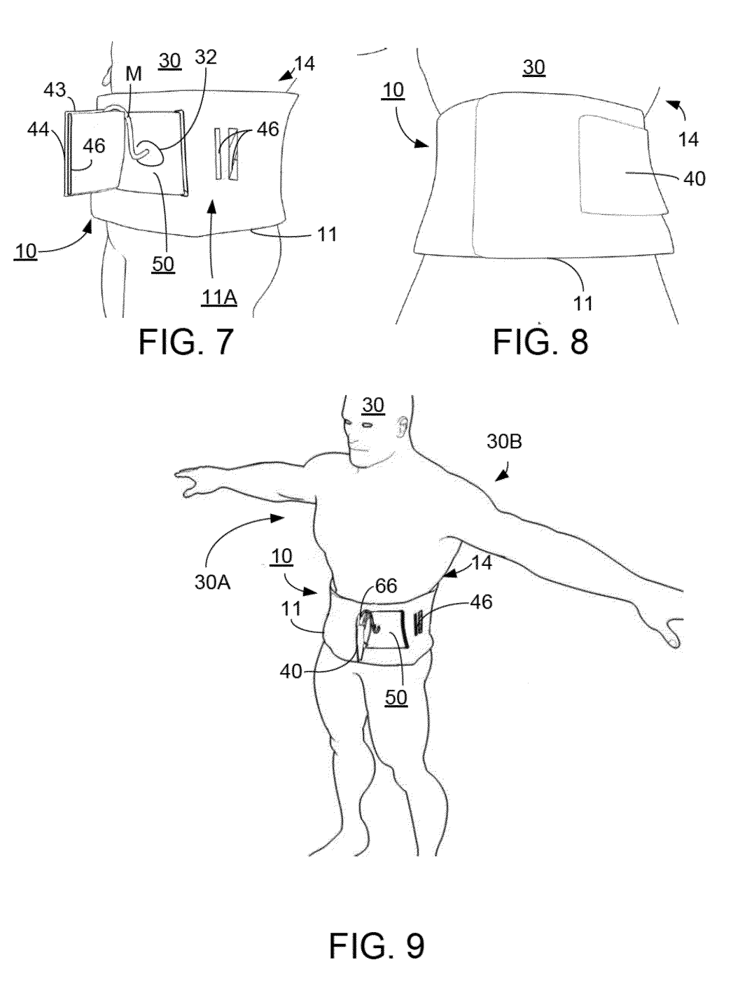

As shown in FIGS. 7, 8 and 9, the wrap 10 is disposed around a portion of a patient's 30 body, e.g. a torso 14 of the patient 30 with both a front or ventral side 30A and back or dorsal side 30B. As shown in FIGS. 8 and 9, a first fastener 20 of the wrap 10 selectively secures the wrap 10 in place about the patient 30. In other embodiments in FIGS. 7 and 8, the fastener 46 is a hook and loop closure located at the end 44 of the flap 40 in an area 11A proximate to the opening 50. Medical paraphernalia M advantageously can access the opening in the wrap 10 oriented to cover a relevant portion of the patient 30 in an insertion point 32 of an area of treatment. Advantages of the various embodiments described in FIGS. 1-13 identify that the wrap 10 may be of various lengths or adjustable in length to surround patients having torsos of varying sizes.

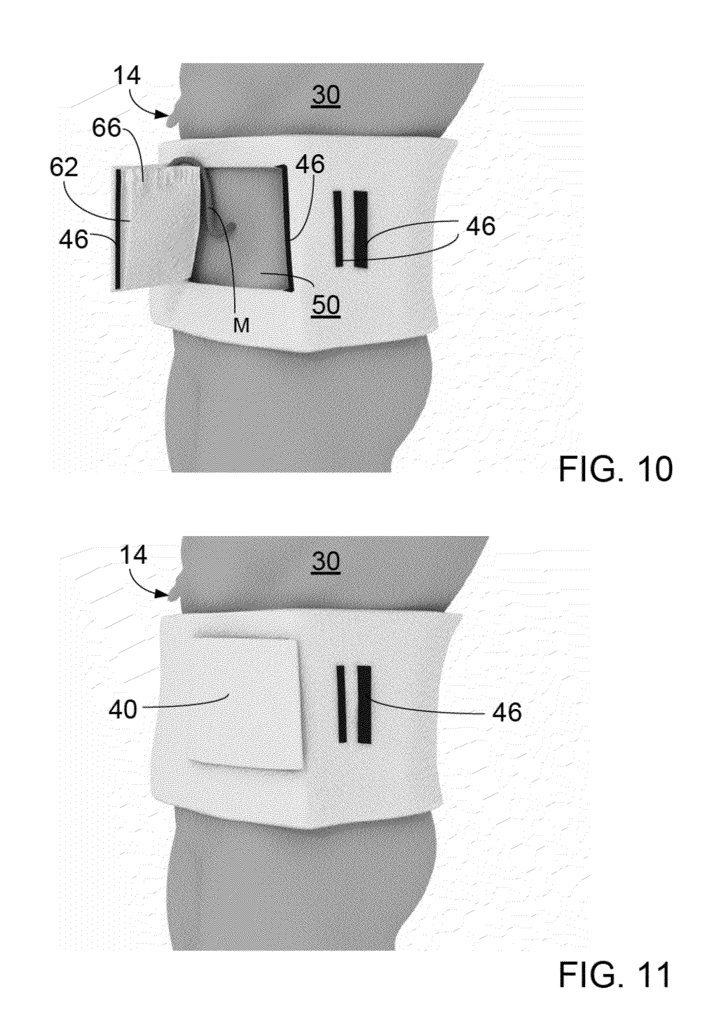

As shown in FIGS. 10 and 11, the wrap 10 is disposed around a portion of a patient's 30 body, e.g. a torso 14 of the patient 30. The flap 40 around a patient's 30 torso 14 opens to an open position, as shown in FIG. 10, to reveal opening 50 and the area of treatment. Medical paraphernalia M advantageously can access the opening 50 in the wrap 10 oriented to cover a relevant portion of the patient 30 in an insertion point 32 of the area of treatment. The flap 40 is adapted to form pouch 60 that includes an expandable wall 62 defining an interior cavity 64 therein. As shown in FIG. 11, the flap 40 is closed to shield the area of treatment by the wrap 10 at times and when necessary for the treatment of the patient. The inner cavity is useful advantageously to organize and store therein various tubes and items adjacent the area for treatment that assists in the patient care.

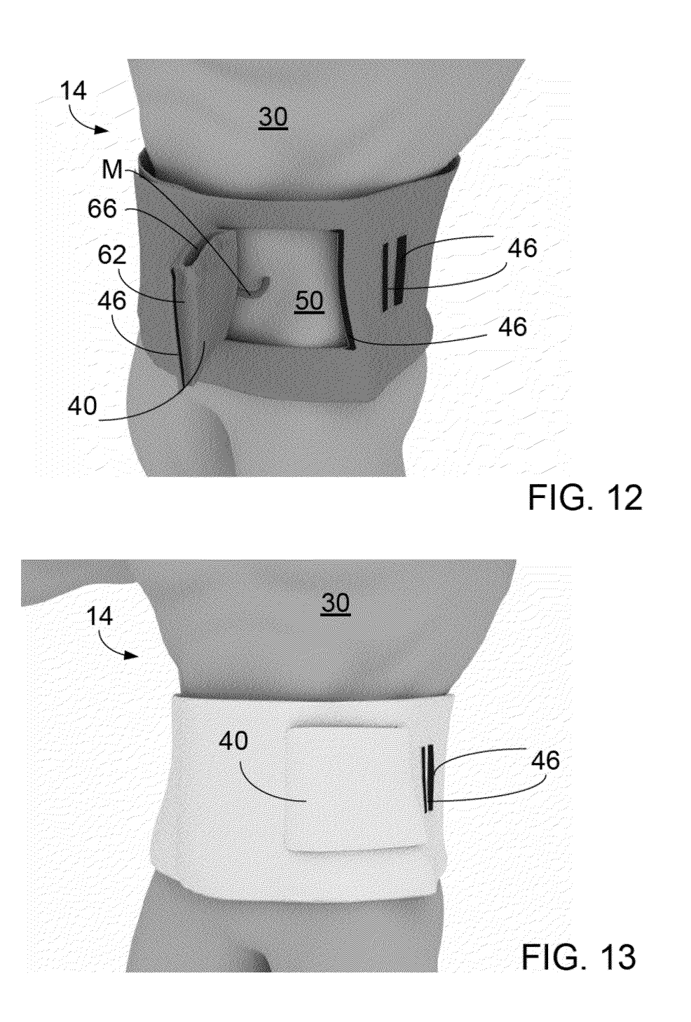

As shown in a perspective view of FIGS. 12 and 13, the wrap 10 is disposed around a portion of a patient's 30 body, e.g. a torso 14 of the patient 30. The flap 40 around a patient's 30 torso 14 opens to an open position, as shown in FIG. 12, to reveal opening 50 and the area of treatment. Medical paraphernalia M advantageously can access through the opening 50 the relevant portion of the patient 30, for example, the insertion point 32 of the area of treatment. The flap 40, as above, is adapted to form pouch 60 that includes an expandable wall 62 defining an interior cavity 64 therein. As shown in FIG. 13, the flap 40 is closed to shield the area of treatment via the wrap 10 at times and when necessary for the treatment of the patient.

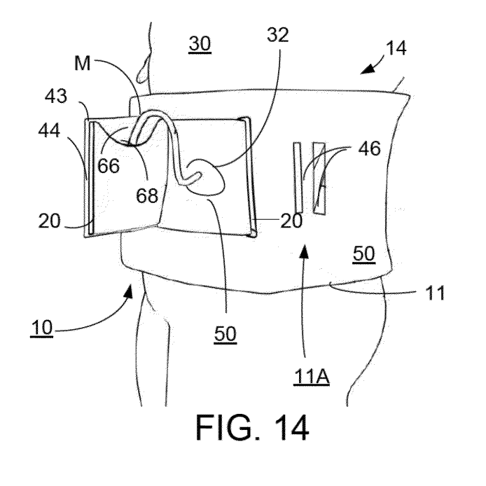

As shown in FIG. 14, the swooped neck feature of a flap 40 of the wrap 10 around a patient's torso 14 in the open or detached position is illustrated. The wrap 10 is disposed around a portion of a patient's 30 body, e.g. a torso 14 of the patient 30 with both a front or ventral side 30A and back or dorsal side 30B. As shown in FIGS. 8 and 9, a first fastener 20 of the wrap 10 selectively secures the wrap 10 in place about the patient 30. In other embodiments in FIGS. 7 and 8, the fastener 46 is a hook and loop closure located at the end 44 of the flap 40 in an area 11A proximate to the opening 50. The opening 66 in the flap 40 has an upper edge 68 adapted to be elongated, a swoop neck, puckering, skirting or otherwise expandable to allow Medical paraphernalia M such as, for example, tubes can access the opening in the wrap 10 oriented to cover a relevant portion of the patient 30 in an insertion point 32 of an area of treatment. The versatile expandable upper edge 68 in the swoop neck is advantageous to not pinch, crimp, inhibit or create blockages in the tube and/or the release or pulling out of a short G-tube, as the expandable upper edge 68 functions to continue to provide support without a tugging effect on the tube.

While certain configurations of structures have been illustrated for the purposes of presenting the basic structures of the present invention, one of ordinary skill in the art will appreciate that other variations are possible which would still fall within the scope of the appended claims. Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A wrap selectively securable about a portion of a patient, the wrap comprising:
a main body comprised of a material that is selectively stretchable;
a first fastener; and
an opening formed in the main body, the opening having
a flap; and
a second fastener selectively securing the flap to the main body.

2. The wrap of claim 1 wherein the flap has a pouch sized to accept at least one of a plurality of medical paraphernalia.

3. The wrap of claim 1 wherein the flap is comprised of a material that is selectively stretchable.

4. The wrap of claim 2 wherein the pouch is comprised of a material that is selectively stretchable.

5. The wrap of claim 2 wherein the pouch has a side wall that is comprised of a material that is selectively stretchable and defines an interior cavity that expands and contracts to accommodate the at least one medical paraphernalia.

6. The wrap of claim 1 wherein the second fastener is a hook-and-loop closure.

7. The wrap of claim 1 wherein the selectively stretchable material is latex-free knit elastic.

8. The wrap of claim 1 wherein one or more of the main body material and the flap are a cotton-polyester-spandex blend.

9. The wrap of claim 1 wherein an outer surface of the main body is comprised of needle-needle-punched cotton felt.

10. The wrap of claim 2 wherein the pouch is comprised of denier nylon taffeta coated with polyurethane.

11. The wrap of claim 1 wherein the main body has one or more ventilating elements disposed therein.

12. The wrap of claim 11 wherein the ventilating elements are comprised of latex-free knit elastic.

13. The wrap of claim 11 wherein the ventilating elements are comprised of wicking polyester material.

14. The wrap of claim 2 wherein the pouch is made of a liquid-resistant material.

15. The wrap of claim 1 wherein the main body has one or more tabs to selectively receive and retain medical paraphernalia.

16. The wrap of claim 1 wherein an outer surface of the main body has one or more tabs to selectively receive and retain medical paraphernalia.

17. The wrap of claim 1 wherein the pouch has an upper edge adapted to be expandable so as to receive and retain medical paraphernalia.
